(19) United States
(12) Patent Application Publication
(10) Pub. No.: US 2012/0184846 A1
(43) Pub. Date: Jul. 19, 2012

(54) IMAGING AND VISUALIZATION SYSTEMS, INSTRUMENTS, AND METHODS USING OPTICAL COHERENCE TOMOGRAPHY

(75) Inventors: Joseph A. Izatt, Raleigh, NC (US); Cynthia A. Toth, Chapel Hill, NC (US); Sina Farsiu, Durham, NC (US); Paul Hahn, Durham, NC (US); Yuankai K. Tao, Durham, NC (US); Justis P. Ehlers, Shaker Heights, OH (US); Justin V. Migacz, Durham, NC (US); Stephanie J. Chiu, Durham, NC (US)

(73) Assignee: DUKE UNIVERSITY, Durham, NC (US)

(21) Appl. No.: 13/353,612

(22) Filed: Jan. 19, 2012

Related U.S. Application Data
(60) Provisional application No. 61/434,242, filed on Jan. 19, 2011.

(51) Int. Cl. A61B 6/00; H04N 7/18; G01B 9/02

(52) U.S. Cl. 600/425; 356/479; 356/72; 348/79

(57) ABSTRACT

Imaging and visualization systems, instruments, and methods using optical coherence tomography (OCT) are disclosed. A method for OCT image capture includes determining a location of a feature of interest within an operative field. The method also includes determining a relative positioning between the feature of interest and an OCT scan location. Further, the method includes controlling capture of an OCT image at a set position relative to the feature of interest based on the relative positioning.

### CROSS REFERENCE TO RELATED APPLICATION

This application claims the benefit of U.S. provisional patent application No. 61/434,242, filed Jan. 19, 2011, the disclosure of which is incorporated herein by reference in its entirety.

### GOVERNMENT RIGHTS NOTICE

This invention was made with government support under grant numbers EY019411 and RR024128, awarded by the National Institutes of Health. The government may have certain rights in the invention.

### TECHNICAL FIELD

The presently disclosed subject matter relates to surgical instruments and imaging equipment, and more specifically, to surgical imaging and visualization systems, instruments, and methods using optical coherence tomography.

### BACKGROUND

Optical coherence tomography (OCT) has emerged as a promising imaging modality for micrometer-scale noninvasive imaging in biological and biomedical applications. Its relatively low cost and real-time, in vivo capabilities have fueled the investigation of this technique for applications in retinal and anterior segment imaging in ophthalmology (e.g., to detect retinal pathologies), early cancer detection and staging in the skin, gastrointestinal, and genitourinary tracts, as well as for ultra-high resolution imaging of entire animals in embryology and developmental biology.

Conventional OCT systems are essentially range-gated low-coherence interferometers that have been configured for characterization of the scattering properties of biological and other samples. By measuring backscattered light as a function of depth, OCT fills a valuable niche in imaging of tissue ultrastructure, and provides subsurface imaging with high spatial resolution (˜1-10 μm) in three dimensions and high sensitivity (>110 dB) in vivo with no contact needed between the probe and the tissue. OCT is based on the one-dimensional technique of optical coherence domain reflectometry (OCDR), also called optical low-coherence reflectometry (OLCR). See Youngquist, R. C., S. Carr, and D. E. N. Davies, Optical Coherence Domain Reflectometry: A New Optical Evaluation Technique. Opt. Lett., 1987. 12: p. 158; Takada, K., et al., New measurement system for fault location in optical waveguide devices based on an interferometric technique. Applied Optics, 1987. 26(9): p. 1603-1606; and Danielson, B. L. and C. D. Whittenberg, Guided-wave Reflectometry with Micrometer Resolution. Applied Optics, 1987. 26(14): p. 2836-2842. In some instances of time-domain OCT, depth in the sample is gated by low coherence interferometry. The sample is placed in the sample arm of a Michelson interferometer, and a scanning optical delay line is located in the reference arm.

The time-domain approach used in conventional OCT has been used in supporting biological and medical applications. An alternate approach involves acquiring as a function of optical wavenumber the interferometric signal generated by mixing sample light with reference light at a fixed group delay. Two methods have been developed which employ this Fourier domain (FD) approach. The first is generally referred to as spectrometer-based or spectral-domain OCT (SDOCT). SDOCT uses a broadband light source and achieves spectral discrimination with a dispersive spectrometer in the detector arm. The second is generally referred to as swept-source OCT (SSOCT) or alternatively as optical frequency-domain imaging (OFDI). SSOCT time-encodes wavenumber by rapidly tuning a narrowband source through a broad optical bandwidth. Both of these techniques can provide improvements in signal-to-noise ratio (SNR) of up to 15-20 dB when compared to time-domain OCT, because SDOCT and SSOCT capture the complex reflectivity profile (the magnitude of which is generally referred to as the “A-scan” data or depth-resolved sample reflectivity profile) in parallel. This is in contrast to time-domain OCT, where destructive interference is employed to isolate the interferometric signal from only one depth at a time as the reference delay is scanned.

Surgical visualization has changed drastically since its inception, incorporating larger, more advanced optics toward increasing illumination and field-of-view (FOV). However, the limiting factor in vitreoretinal surgery remains the ability to distinguish between tissues with subtle contrast, and to judge the location of an object relative to other retinal substructures. S. R. Virata, J. A. Kylstra, and H. T. Singh, Retina 19, 287-290 (1999); E. Garcia-Valenzuela, A. Abdelsalam, D. Eliott, M. Pons, R. Iezzi, J. E. Puklin, M. L. McDermott, and G. W. Abrams, Am J Ophthalmol 136, 1062-1066 (2003). Furthermore, increased illumination to supplement poor visualization is also limited by the risks of photochemical or photothermal toxicity at the retina. S. Charles, Retina 28, 1-4 (2008); J. R. Sparrow, J. Zhou, S. Ben-Shabat, H. Vollmer, Y. Itagaki, and K. Nakanishi, Invest Ophthalmol Vis Sci 43, 1222-1227 (2002). Finally, inherent issues in visualizing thin translucent tissues, in contrast to underlying semitransparent ones, require the use of stains such as indocyanine green, which is toxic to the retinal pigment epithelium. F. Ando, K. Sasano, N. Ohba, H. Hirose, and O. Yasui, Am J Ophthalmol 137, 609-614 (2004); A. K. Kwok, T. Y. Lai, K. S. Yuen, B. S. Tam, and V. W. Wong, Clinical & experimental ophthalmology 31, 470-475 (2003); J. Lochhead, E. Jones, D. Chui, S. Lake, N. Karia, C. K. Patel, and P. Rosen, Eye (London, England) 18, 804-808 (2004).

SDOCT has demonstrated strong clinical success in retinal imaging, enabling high-resolution, motion-artifact-free cross-sectional imaging and rapid accumulation of volumetric macular datasets. N. A. Nassif, B. Cense, B. H. Park, M. C. Pierce, S. H. Yun, B. E. Bouma, G. J. Tearney, T. C. Chen, and J. F. de Boer, Optics Express 12, 10 (2004); M. Wojtkowski, V. J. Srinivasan, T. H. Ko, J. G. Fujimoto, A. Kowalczyk, and J. S. Duker, Optics Express 12, 2404-2422 (2004). Current generation SDOCT systems achieve greater than 5 μm axial resolutions in tissue, and have been used to obtain high resolution datasets from patients with neovascular AMD, high risk drusen, and geographic atrophy. M. Stopa, B. A. Bower, E. Davies, J. A. Izatt, and C. A. Toth, Retina 28, 298-308 (2008). Other implementations of OCT including SSOCT may offer similar performance advantages.

Intraoperative guidance of surgical procedures using optical coherence tomography (OCT) holds promise to aid surgeons visualize microscopic tissue structures in preparation for and during surgery. This potential includes visualization of the critical interface where surgical tools (e.g., scalpels, forceps, needles, scrapers) intersect and interact with tissue surfaces and sub-surface structures. In many cases, critical aspects of this dynamic interaction exceed the spatial or temporal resolution of conventional imaging devices used during surgery (e.g., surgical microscopes, endoscopes, ultrasound, CT, and MRI). A particularly compelling case for OCT guidance is in ophthalmic surgery, since OCT is already a widely accepted diagnostic in ophthalmology, and real-time visualization of delicate and translucent tissues during intrasurgical maneuvers could be of great benefit to surgeons and patients. In ophthalmic surgery of both anterior and posterior segments of the eye, the typical imaging modality used to visualize microscopic tissue structures is stereo zoom microscopy. Surgical microscopes provide real-time natural color imaging to the surgeon, however the quality of the imagery is often severely limited by the available illumination and the quality of the patient eye's own optics, particularly for retinal surgery. Additionally, conventional surgical microscopy only provides en-face imagery of the surgical field, which bears little to no depth information, forcing the surgeon to infer when instruments are in contact with tissue surfaces, how deep instruments are penetrating, how thick tissue structures are, etc. As a cross-sectional imaging modality, OCT is particularly well suited to providing critical depth-resolved information in ophthalmic surgery. The advent of Fourier domain OCT (FDOCT) approaches including both SDOCT and SSOCT is especially promising for providing real time feedback because of their enhanced SNR compared to prior time-domain. OCT methods, thus enabling much faster imaging than previously available.

FDOCT systems have been developed for use during surgery, including breast cancer biopsy and surgery and ophthalmic surgery of the anterior segment and retina. High speed FDOCT systems are available, including research SSOCT systems now operating in excess of 5,000,000 A-scans/sec, corresponding to tens of volumes per second. However, these systems are very complex and expensive, and require illumination light levels which may not be safe for human ocular exposure. Even with advances in higher speed OCT scanning, real-time complete volumetric imaging in the intrasurgical setting, where OCT imaging must be safely compatible with other bright light sources, may not be achievable. Thus, there is a need for systems, equipment, and techniques for using current or near-future generation OCT systems to provide useful, real time feedback to the surgeon.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Disclosed herein are microscope-integrated OCT (MIOCT) systems that integrate OCT imaging into the optical path of a surgical microscope for direct and OCT imaging. As a result, fast feedback is provided to the surgeon with less intrasurgical disruption than previous intrasurgical OCT protocols that require the surgeon to use a hand-held OCT probe during intermittent pauses during surgery. According to an aspect, an MIOCT system utilizes a standard current-generation SDOCT engine which images at a rate of 15,000-40,000 A-scans/sec, ˜20 B-scans/sec (with 1000 A-scans/B-scan), and acquires a complete 3D volumetric image in ˜5 sec. The systems and methods disclosed herein provide useful, near real-time intrasurgical imaging by using current or near-future generation OCT hardware in combination with a feedback control system to localize OCT image data acquisition to the region of the tip of the surgical tool, or the site of its interaction with the tissue. This occurs by tracking some predetermined feature of the surgical tool (such as its tip, some markings made upon it or indented into it, or a reflective spot or light source located on it) and using that position information to direct the OCT system to concentrate image acquisition in a region relative to that position which is of anticipated importance during surgery. A variety of OCT scan patterns, protocols, and displays are disclosed, which may be of particular value for guiding surgery, such as small numbers of B-scans (which can still be acquired in real time as perceived by the surgeon) acquired with specific orientation relative to the tool tip, small volumetric datasets (which can still be acquired in real time as perceived by the surgeon) localized to the tool tip location, or other novel combinations of A-scans. In addition to providing real-time imaging capability, the image data thus acquired can also be used in combination with the saved instrument track data and image processing techniques to build up and maintain an evolving three-dimensional (3D) rendition of the entire operative field of view. The control system may also perform adaptive sampling of the field of view, for example directing the OCT scanner to prioritize filling in missing information when tool movement reveals a previously unsampled region of retina, which had until then been shadowed by the tool. Thus, in this disclosure, an intelligent feedback control system is disclosed that can be readily modified to the surgical style of the surgeon.

According to an aspect, a method for OCT imaging includes receiving multiple OCT B-scans of a field of view area that includes an instrument. The method also includes applying spatial compounding to the B-scans to generate an OCT image of the area of the field of view area.

According to another aspect, a method for OCT image capture includes determining a location of a feature of interest within an operative field. The method also includes determining a relative positioning between the feature of interest and an OCT scan location. Further, the method includes controlling capture of an OCT image at a set position relative to the feature of interest based on the relative positioning.

According to another aspect, a surgical microscope system includes a heads-up display (HUD). The system also includes an ocular eyepiece unit having the HUD integrated therein for display via the ocular eyepiece unit. Further, the system includes a user interface controller configured to determine surgical information associated with a surgical site image projected for view through the ocular eyepiece unit. The user interface controller is also configured to control the HUD to display the surgical information.

According to yet another aspect, a surgical instrument for use in optical coherence tomography (OCT)-imaged surgical procedures is disclosed. The surgical instrument comprises a body having a predefined shape for improving capture of OCT images of nearby tissue during a surgical procedure, or being made from a combination of materials which optimize its appearance in OCT or video images or reduce the amount by which the instrument shadows the underlying tissue in OCT imaging.

### BRIEF DESCRIPTION OF THE DRAWINGS

The foregoing summary, as well as the following detailed description of various embodiments, is better understood when read in conjunction with the appended drawings. For the purposes of illustration, there is shown in the drawings exemplary embodiments; however, the presently disclosed subject matter is not limited to the specific methods and instrumentalities disclosed. In the drawings:

FIG. 1 is a schematic diagram of the microscope-attached components of an example microscope-integrated OCT (MIOCT) system according to embodiments of the presently disclosed subject matter;

FIG. 2 is a schematic diagram of an OCT unit and a microscope having a vitreoretinal viewing unit;

FIG. 3 is a schematic diagram of an example microscope-integrated OCT (MIOCT) system in accordance with embodiments of the present disclosure;

FIG. 4 is a flowchart of an example method for OCT imaging in accordance with an embodiment of the present disclosure;

FIGS. 5A-5F illustrate various B-scan patterns with respect to a surgical instrument in accordance with embodiments of the present disclosure;

FIG. 6 is an image of a volumetric dataset acquired of an eye by scanning the general location of instrument-tissue interaction and associated B-scans;

FIG. 7 shows a time lapse and lateral position montage of sequentially acquired volume images of intrasurgical manipulations using OCT and spatial compounding in accordance with embodiments of the present disclosure;

FIG. 8 is a flowchart of an example method for tracking a feature of interest using OCT in accordance with embodiments of the present disclosure;

FIG. 9 illustrates a schematic diagram of an example model for image feature tracking in accordance with an embodiment of the present disclosure;

FIG. 10 illustrates images in which two sparsely sampled sequences are fused together creating a dense representation of the underlying pathological structures;

FIG. 11 depicts various images of example data obtained from simulated surgery on an excised porcine eye with the anterior segment removed; and

FIG. 12 illustrates orthogonal B-scans acquired using the scan pattern shown in FIG. 5C during simulated porcine surgery and orthogonal B-scans.

### DETAILED DESCRIPTION

The presently disclosed subject matter is described with specificity to meet statutory requirements. However, the description itself is not intended to limit the scope of this patent. Rather, the inventors have contemplated that the claimed subject matter might also be embodied in other ways, to include different steps or elements similar to the ones described in this document, in conjunction with other present or future technologies.

Like numbers may refer to like elements throughout. In the figures, the thickness of certain lines, layers, components, elements or features may be exaggerated for clarity.

The terminology used herein is for the purpose of describing particular embodiments only and is not intended to be limiting of the presently disclosed subject matter. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Further, it is noted that although the term “step” may be used herein to connote different aspects of methods employed, the term should not be interpreted as implying any particular order among or between various steps herein disclosed unless and except when the order of individual steps is explicitly described.

Unless otherwise defined, all terms (including technical and scientific terms) used herein have the same meaning as commonly understood by one of ordinary skill in the art to which the present subject matter belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The presently disclosed subject matter is described below with reference to block diagrams and/or flowchart illustrations of methods, apparatus (systems) and/or computer program products. It is understood that each block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus to produce a machine, such that the instructions, which execute via the processor of the computer and/or other programmable data processing apparatus, create means for implementing the functions/acts specified in the block diagrams and/or flowchart block or blocks.

In some embodiments, OCT imaging of a surgical specimen or operating field in conjunction with standard surgical microscopy may be used to give the surgeon an additional, two- or three-dimensional view of structures which may be difficult or impossible for the surgeon to visualize with a standard microscope alone. These structures may be difficult to visualize because they are beyond the resolution limit of the microscope optics or of the surgeon's eye, or are poorly lit, translucent, opaque, or buried in a translucent or opaque structure. OCT 2D images may be acquired in a cross-sectional view which complements the en-face view which the surgeon sees through the surgical microscope. OCT 3D volume images convey more information regarding the structures and their spatial orientation and relationships than is available in the surgeon's standard view through the microscope.

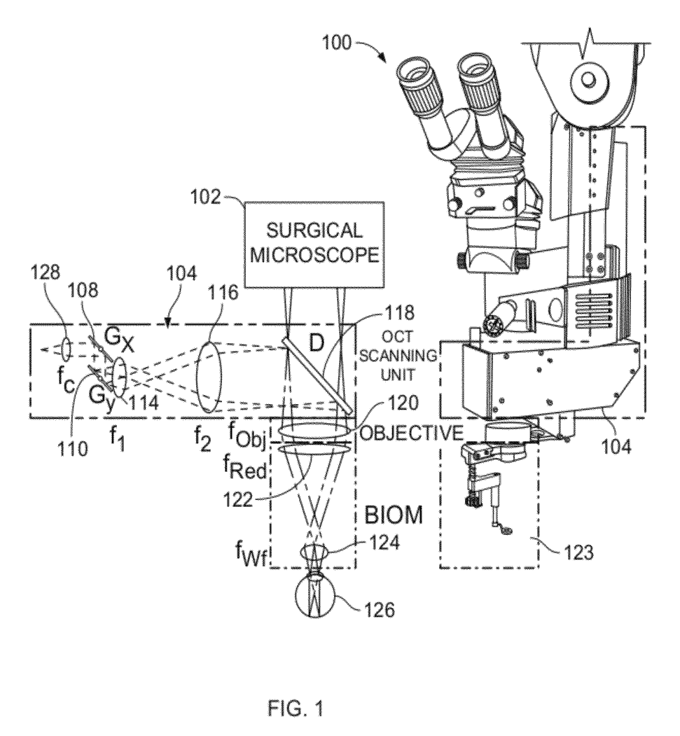

FIG. 1 illustrates a schematic diagram of the microscope-attached components 100 of an example microscope-integrated OCT (MIOCT) system according to embodiments of the presently disclosed subject matter. Referring to FIG. 1, the system is implemented as an attachment to an ophthalmic surgical microscope 102 with a binocular indirect ophthalmo-microscope (BIOM) attachment for imaging the retina. The microscope-attached components 100 include an OCT scanning unit 104 which is part of the sample arm of an OCT system. The OCT scanning unit 104 includes a collimating lens 128, two-axis galvanometer scanners (Gₓ, G_{y}) 108, 110, a beam-forming assembly comprising a 12.5× beam expander 114, 116 (f₁, f₂), and a beamsplitter 118. The OCT scanning unit 104 may direct an OCT beam through a microscope objective 120 shared with the surgical microscope, and a BIOM retinal viewing attachment including a reduction lens 122 and a non-contact wide-field lens 124, in order to scan the OCT beam across a 12 mm field of view on a retina of a patient's eye 126 or another surgical site. The backscattered OCT light may be returned back through the same optical path and re-focused by the collimating lens 128 into an optical fiber which conveys the light back to the OCT interferometer. The BIOM unit is just one option for converting a standard operating room microscope for retinal viewing, the other primary means used in retinal surgery is imaging through a contact lens designed to compensate for the optical power of the cornea. Both the BIOM unit and contact lens are for conversion of a standard surgical microscope for retinal imaging. Without such conversion, a standard surgical microscope may be used for imaging of the anterior segment of the eye or for any other exposed tissue surface in the body. All of the same methods for OCT scanning through the BIOM for retinal imaging may equivalently be accomplished through a contact lens for retinal imaging, or with no other optics between the shared objective and the tissue surface for non-retinal imaging. Although not shown in FIG. 1, it is noted that the MIOCT system may also include an OCT engine and computer as described herein, such as the computer and OCT engine shown and described with respect to FIG. 3.

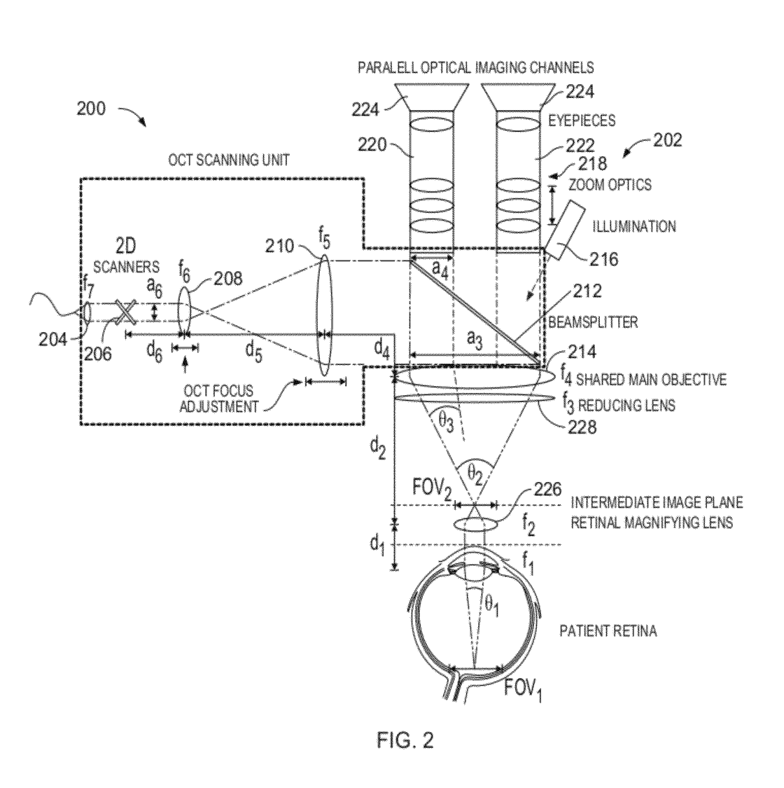

FIG. 2 illustrates an OCT scanning unit 200 and a microscope 202 having a vitreoretinal viewing unit. The OCT scanning unit 200 includes a collimating lens 204, scanners 206, a beam forming optical assembly having optics or lenses 208, 210, and a beamsplitter 212. The microscope 202 includes a shared main objective 214, an illumination unit 216, microscope optics 218 having dual channels 220, 222 and respective eyepieces 224. The vitreoretinal viewing unit includes a non-contact widefield lens 226 and a reduction lens 228. In this configuration, OCT may be incorporated into an intrasurgical microscope for obtaining OCT images of the retina and macula with increased resolution and brightness supported by the physical dimensions of the microscope, while maintaining compatibility for matching the field of view of the OCT scanning unit 200 and microscope views and avoiding OCT image vignetting. The OCT sample arm beam is introduced into the optical path of the microscope in the infinity space above the shared main objective 214 of the microscope, by use of a dichroic beamsplitter 212 which reflects OCT wavelengths (typically in the near infrared) while transmitting visible wavelengths carrying the white light microscope image to the optical imaging channels above. Such a dichroic mirror is typically referred to as a “hot mirror.” The OCT sample arm beam is assumed to be delivered to the vicinity of the microscope via a single mode optical fiber and impinging upon a collimating lens 204 with focal length f₇, as depicted in FIG. 2, or it may impinge directly from a bulk-optic OCT system lacking optical fibers. The narrow OCT sample arm beam is incident on a pair of orthogonal optical scanners 206, depicted as a bolded cross in FIG. 2. Many different scanning technologies can and have been used in OCT systems, although the most typical are limited-angle galvanometer scanners. Most scanning technologies however share the attributes that their cost and performance is typically inversely proportional to the size of their optical aperture; this is why they are typically employed in OCT systems where the sample arm beam is narrow. In FIG. 2, an orthogonally oriented pair of scanners is indicated, capable of scanning the narrow OCT sample arm beam over a limited range of angles in two dimensions under computer control. It will be understood to one skilled in the art of optical scanning systems that such a pair may be constructed from a single mirror having the capability of pivoting in two dimensions, or a pair of single-axis galvanometer scanners located in close proximity (which is the most common arrangement), or a pair of single-axis galvanometer scanners with the optical aperture of one being imaged into the optical aperture of the other using, for example, a standard 4f optical imaging system comprised of a pair of lenses. It is also to be understood that any of these technologies may be used and equivalently represented by the cross symbol depicted in FIG. 2.

As illustrated, the beam-forming optical assembly is positioned between the 2D scanners 206 and the OCT beamsplitter 212 above the shared main objective 214 of the microscope 202. The design purpose of the optical assembly is to match the size, divergence, and scanning geometry of the OCT sample arm beam to the maximum capabilities supported by the microscope 202. In FIG. 2, this is accomplished using a simple, adjustable Keplerian telescope as a beam expander which is placed at the proper position with respect to the microscope, although other possible optical designs could be used to accomplish the same objective (such as, most simply, a Galilean teslescope). For the beam former depicted in FIG. 2, the optical design parameters are as follows. The ratio of the focal lengths of the two lenses 210 (f₅) and 208 (f₆) may be chosen so that the narrow OCT beam with 1/e² beam diameter a₆ is expanded to approximately fill the back aperture of the shared main microscope objective a₃. This is accomplished when:

$$\frac{{f}_{5}}{{f}_{6}} = \frac{{a}_{3}}{{a}_{6}} . \qquad Eq . ( 10 )$$

In order that the divergence of the OCT sample beam generally match the divergence of the microscope image light above the shared objective which may not be exactly parallel, and therefore provide the capability to closely match the position within the patient's retina of the OCT focus to that of the microscope, some variability should be provided in the position of either or both of lenses 210 (f₅) and 208 (f₆), as depicted in FIG. 2. This can be accomplished by mounting one or both lenses on a mechanical stage which can be adjusted manually or electronically by the surgeon or an assistant while viewing both images. For this purpose, an adjustment range of either lens of approximately 5% of its focal length will suffice.

In order to reduce or prevent vignetting of the OCT beam during scanning, while simultaneously making use of the entire optical aperture of the shared objective at the same time, optimal design of the beam-forming optical assembly includes provision that the OCT beam pivot through the main objective rather than scan across it. In general, this can be accomplished by designing the optical assembly in such a way as to optically image the optical scanner plane into the main objective back aperture. For the Keplerian telescope depicted in FIG. 18, this is accomplished by placement of the lenses of the telescope relative to the scanner and objective location, such that the following relationship among the distances between the lenses is satisfied:

d₆=f₆; dₛ=fₛ+f₆; d₄=fₛ Eq. (1)

Furthermore, for OCT imaging of a patient's retina with increased or maximum resolution and image brightness, the OCT sample arm beam may be designed at the position of the patient's cornea to have a beam diameter which is the maximum over which a human's eye is approximately diffraction limited (typically 2-3 mm without use of adaptive optics, and up to 6-7 mm if adaptive optics are used to compensate for the eye's aberrations), and that the scanning OCT beam pivot through the patient's iris plane rather than scan across it, so that the entire available optical aperture is used for all scan widths without vignetting. The first condition on beam size at the cornea is satisfied by realizing that the lenses 226 (f₂), 228 (f₃), and 214 (f₄) operate essentially as a Keplerian beam reducer. As a simplifying assumption, the reducing lens 228 (f₃) typically has much less optical power than the main objective 214 (f₄), and is located directly adjacent to it. Then, these two lenses can be considered as operating together as a single lens, with optical power given by the sum of the optical powers of the lenses individually, and located at the position of the main objective 214. According to this approximation, the main objective 214 is replaced by a modified main objective with focal length f₄ according to:

$$\frac{1}{{f}_{4}^{′}} = \frac{1}{{f}_{3}} + \frac{1}{{f}_{4}} \qquad Eq . ( 2 )$$

Now the design condition on the choice of lenses f₂ and f₄′ to ensure the correct beam size on the patient's cornea is given by:

$$\frac{{f}_{4}^{′}}{{f}_{2}} = \frac{{a}_{3}}{{a}_{1}} , \qquad Eq . ( 3 )$$

where a1 is the desired collimated beam size on the cornea. Finally, in order to have the OCT beam pivot through the patients' iris plane rather than scan across it, the position of lens f₂ should be set so that it forms a real image of the aperture of the shared main objective at the location of the patient's iris plane. Thus, the distances d₁ and d₂ (which equals f_{4′}+f₂) should be set according to:

$$\frac{1}{{f}_{2}} = \frac{1}{{f}_{2} + {f}_{4}^{′}} + \frac{1}{{d}_{1}} \qquad Eq . ( 4 )$$

As a practical design procedure, f₂ and f₃ should be chosen according to Eq. (2) and (3) given the constraint the microscope imposes on f₄, then d₁ may be chosen according to Eq. (4).

In the foregoing, it is to be understood that the distances d₄, d₅, etc. correspond to the center-to-center distances between the lenses referred to. Also, it is to be understood that the relationships given in all of these design equations are simplified according to the assumption that all of the lenses act as ideal lenses which is appropriate for first-order design. For production design, professional lens design software may be used to optimize higher order performance, however following the principles described here.

In the configuration illustrated in FIG. 2, all of the OCT optical elements are generally positioned either within the microscope body itself, or else sufficiently high up on the microscope body that the OCT assembly will not interfere with the surgeon's view of or access to the patient. However, this configuration may lengthen the overall height of the surgical microscope by displacing the optical imaging channels upward in order to make room for the beamsplitter, which may be inconvenient for the surgeon. Secondly, the configuration illustrated in FIG. 2 utilizes the main objective for OCT in addition to white light imaging.

FIG. 3 illustrates a schematic diagram of an example microscope-integrated OCT (MIOCT) system 300 in accordance with embodiments of the present disclosure. Referring to FIG. 3, the system 300 includes the microscope 202, the OCT scanning unit 200, a video fundus camera 302, and one or more computers 304. In this example, the microscope 202 is configured with a port for the video fundus camera 302, which may capture video of a surgical site 306, which is a retina in this example. The view of the surgical site 306 captured by the camera 302 may be the same as that viewed by an operator of the microscope 202 through oculars 308. In an example, the camera 302 may be a 1.3 megapixel CMOS color camera configured to digitize the top-down view of the surgical site 306. Further, in this example, an intensity peak-finding algorithm may be implemented by the computer 304 for capturing images of the surgical site 306 and a surgical instrument positioned within a field of view. The frame rate of the camera 306 may operate at 15 frames per second (fps) that is comparable to a B-scan rate of the OCT unit 200. The surgical instrument may be a diamond-dusted silicone-tipped instrument (e.g., a Tano scraper) having a non-reflective tip, for example, and marked with a predefined color marking for tracking in accordance with embodiments of the present disclosure. An OCT engine 307 may interface the OCT scanning unit 200 with the computer 304 and may include, but is not limited to, a light source, an interferometer, a reference arm, and a detector. In the case of an OCT engine based on SDOCT technology, the light source may be a broadband light source such as a superluminescent diode, and the detector may be a spectrometer coupled to a linear detector array. In the case of an OCT engine based on SSOCT technology, the light source may be a frequency swept laser and the detector may be a single or multiple receivers arranged in a dual-balanced configuration.

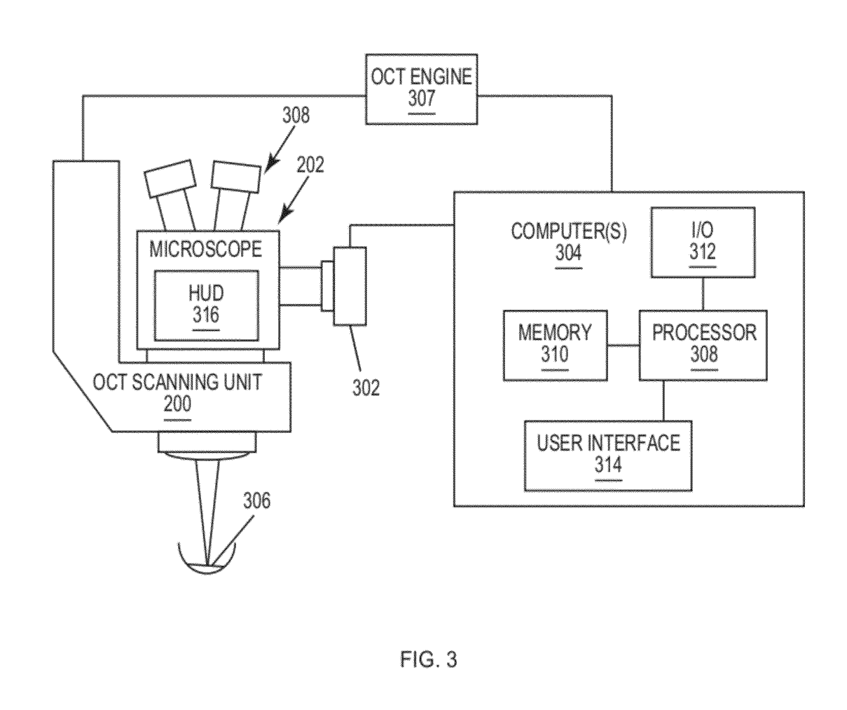

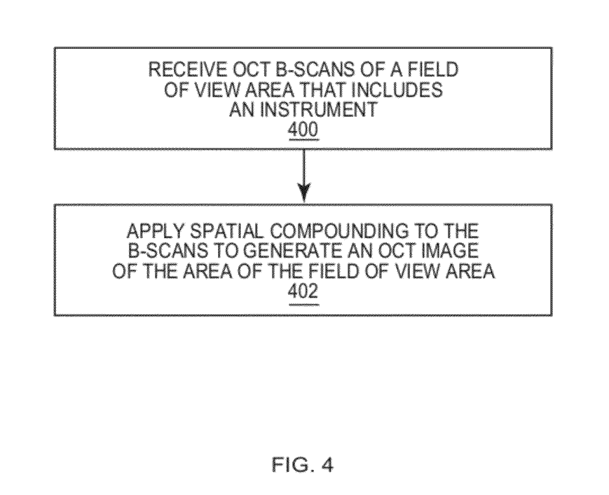

The computer 304 may be any suitable computing device and include a processor 308 and memory 310 for implementing surgical imaging and visualization of a surgical site and instruments using OCT in accordance with embodiments of the present disclosure. In accordance with an embodiment, FIG. 4 illustrates a flowchart of an example method for OCT imaging. The method of FIG. 4 is described in the following example as being implemented by the system of FIG. 3; however, it should be understood that the method may be implemented by any suitable system. In this example, the method includes receiving OCT B-scans of a field of view area that includes an instrument (step 400). For example, the B-scans captured by the OCT unit 200 may be communicated to the computer 304. The computer 304 may include an input/output (I/O) module 312 configured to receive the B-scans for storage in memory 310 and processing by the processor 308. A-scans or any other suitable images may be captured by the OCT unit 200 and communicated to the computer 304. Example surgical instruments include, but are not limited to, scalpels, scrapers, and forceps.

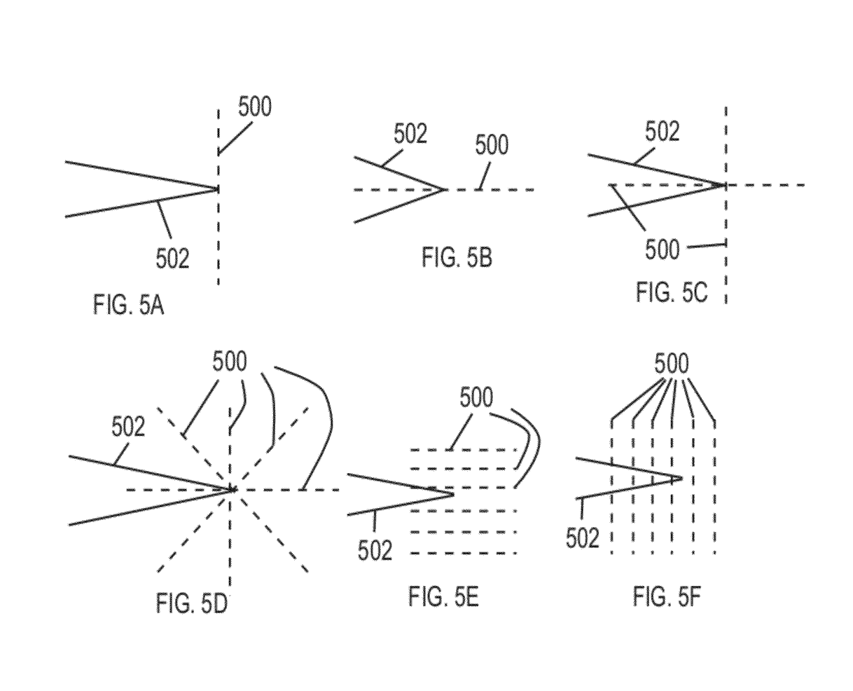

The computer 304 may operate as a controller for controlling a timing and scan pattern of scans captured by the OCT unit 200. The computer 304 may control the OCT unit 200 to capture scan patterns based on a surgical tool position and orientation. For example, the scan pattern of the B-scans may be substantially aligned with an axis of the surgical instrument. In an example, the B-scans may have a pattern with a long-axis aligned substantially with an axis of the instrument and a short-axis substantially collinear with the surgical instrument. FIGS. 5A-5F illustrate various B-scan patterns, indicated by broken lines 500, with respect to a surgical instrument 502 in accordance with embodiments of the present disclosure. Referring to FIGS. 5A and 5B, B-scans can be acquired in real time in orientations either perpendicular as shown in FIG. 5A or parallel as shown in FIG. 5B with respect to an axis of the surgical instrument 502. In another example, FIG. 5C shows a rapidly captured set of orthogonal B-scans. In another example, the orientation of the B-scans may be arbitrary based on a preference of a surgeon or a type and/or shape of the surgical instrument. In yet another example, two B-scans may be oriented directly orthogonal and intersecting at the middle, and with one B-scan being directly aligned with an axis of the surgical instrument and the other B-scan being perpendicular. These B-scans can provide desired information about the instrument angle along or relative to tissue, as well as have a second view (e.g., perpendicular view) such that the surgeon has a different perspective. FIGS. 5E and 5F show scan patterns in which small volumes may be acquired with a fast-axis either parallel (FIG. 5E) or perpendicular (FIG. 5F) to the instrument axis. Also, scanning the OCT beam along the tool initially, and subsequently scanning directly beside the instrument may be useful because the scanning beam may not be obstructed by the partially or fully opaque instrument.

Referring again to FIG. 4, the method includes applying spatial compounding to the B-scans to generate an OCT image of the area of the field of view area (step 402). For example, spatial compounding may include combining the B-scans to generate an OCT image. The B-scans may be combined to generate the OCT image by averaging the B-scans. In another example, the B-scans may be combined to generate the OCT image by performing a weighted average of the B-scans. The processor 308 and memory 310 of the computer 304 shown in FIG. 3 may implement the step of spatial compounding. The generated OCT image may subsequently be displayed by control of a display of a user interface 314. The OCT unit 200 may be controlled to capture OCT B-scans at a lateral spacing of a predetermined distance.

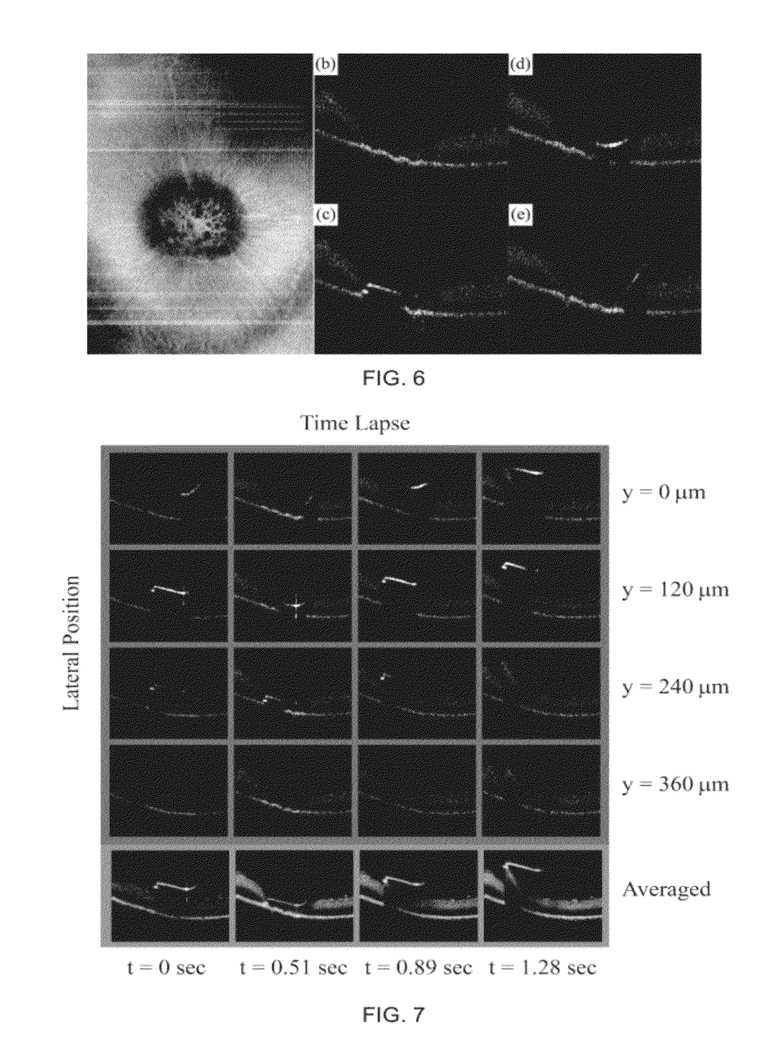

FIG. 6 is an image (a) of a volumetric dataset acquired of an eye by scanning the general location of instrument-tissue interaction. The image capture occurred over an area of 8×0.4 mm. The area was sampled with 1024×256 pixels (spectral×lateral) at 20 kHz line-rate, and with 10 B-scans across the short scanning axis as shown in image (a) of FIG. 6. The pattern of the scan is indicated by the broken arrows shown in image (a) of FIG. 6. The long-axis of the scan pattern was positioned approximately along the axis of a surgical instrument, and the short-axis of the scan pattern was approximately collinear with the surgical instrument. Images (b)-(e) shown in FIG. 6 represent B-scans, separated by 0.12 mm across the short-axis of the scan patter, demonstrating the instrument cross-section moving into and out of view. The scans of images (b)-(e) show regions of tissue deformation and discrete portions of the surgical instrument, which is a subretinal needle in this example, that overlap with the OCT field of view. In spatial compounding, all of the 10 B-scans in the short scanning axis are averaged to allow for visualization of the complete instrument and underlying tissue without the sectioning and shadowing artifacts which are apparent in the individual B-scans. In this example, the long scanning axis is large enough such that any instrument motion remains at a temporal spacing of 25.6 ms per frame, or at a frame rate of 39 Hz. Here, cross-sections of intrasurgical instrument-tissue interactions acquired at video rates over the entire region of interest are shown. As an additional benefit, spatial compounding may serve to improve signal-to-noise (SNR) of the images by averaging a series of B-scans.

FIG. 7 shows a time lapse and lateral position montage of sequentially acquired volume images of intrasurgical manipulations using OCT and spatial compounding in accordance with embodiments of the present disclosure. Along the lateral position direction, for each time point, evenly spaced raw B-scans across the short scanning axis are shown in the top four rows of frames in the vertical axis to illustrate inadequate visualization of the entire instrument and its interaction with the tissue by sampling at a single lateral position. Depending on the orientation of the instrument relative to the scanning axis, only portions of the instrument overlap with the field of view of the OCT in each B-scan, and the instrument moves relative to the OCT field of view over time (see the top four rows of frame in the horizontal axis). All 10 B-scans across the short scanning axis may be averaged (see the bottom row of frames) to demonstrate the advantages of spatial compounding for visualizing the entire region of interest. In this example, the surgical site is a porcine eye which was lifted using a subretinal needle and imaged using MIOCT. The surgical procedure was performed by viewing through a surgical microscope with concurrent acquisition of MIOCT volumes. A 8×0.4 mm volumetric dataset was acquired with 10 B-scans, sampled with 1024×256 pixels (spectral×lateral) at 20 kHz line-rate. The long-axis of the scan pattern was positioned approximately along the axis of the instrument and the short-axis of the scan pattern was approximately collinear with the instrument. 100 consecutive volumes were then acquired to capture tissue-instrument interactions. Here, individual raw B-scans are shown in the frames of the top 4 rows for different time points at evenly spaced lateral positions across the short scanning axis. These individual frames show incomplete visualization of the surgical tool as the instrument cross-section moves into and out of the interrogation path of the OCT beam. By averaging these raw frames across all 0.4 mm of the short scanning dimension using spatial compounding, complete instrument cross-sections can be visualized for all time points in the dataset shown in the bottom row of frames. Over the course of the time lapse, these averaged frames show retinal deformation due to compression of the vitreous by the subretinal needle and to lifting of the retinal membrane by the subretinal needle. Given the sampling parameters used in this example acquisition, the temporal spacing between each average frame is 25.6 ms, resulting in an effective spatial compounding frame rate of 39 Hz, demonstrating video rate interrogation of intrasurgical manipulations. These sampling parameters can be optimized to specific region of interest sizes and spatial and temporal sampling densities.

In accordance with embodiments of the present disclosure, methods for tracking OCT image capture to the location of a feature of interest in an operative field are provided. As an example, such tracking methods may be used to track a location of a feature of interest within one or more captured images. The images may be captured, for example, by the camera 302 shown in FIG. 3. Alternatively, for example, the images may be OCT summed voxel projection (SVP) images, OCT B-scans, or a scanning laser ophthalmoscopy (SLO) image. Further, the captured images may be stored in the memory 310 of the computer 304. The feature of interest may be identified and tracked for controlling the subsequent capture of OCT images such that these images include the feature of interest. The feature of interest may be, for example, a surgical image, tissues, or portions thereof. Alternatively, the location of the feature of interest within the surgical field which is desired to be tracked with OCT may be obtained through means independent of any optical imaging modality. Such means may include localization of features or instruments by alternative imaging modalities such as ultrasound, computed tomography, or magnetic resonance imaging (including microscopic versions of each modality), or by non-imaging means such as instrument tracking via radiofrequency triangulation.

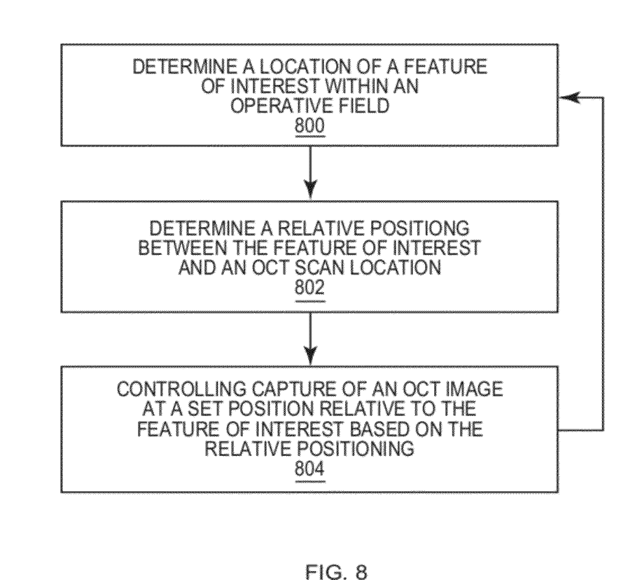

FIG. 8 illustrates a flowchart of an example method for tracking a feature of interest using OCT in accordance with embodiments of the present disclosure. The method of FIG. 8 is described in the following example as being implemented by the system of FIG. 3; however, it should be understood that the method may be implemented by any suitable system. In this example, the method includes determining a location of a feature of interest within an operative field (step 800). For example, images may be captured that include a feature of interest. Example images include, but are not limited to, OCT images, such as B-scans, SVP images, or SLO images, captured by the OCT unit 200 shown in FIG. 3. In another example, the captured images may be videos captured by the camera 302. The images may be communicated to the computer 304.

A feature of interest may be, for example, a surgical instrument or a tissue feature within a surgical site view. In an example, a surgical instrument may include a straight edge, a color, a recognizable shape of an area of a surgical instrument, a homogenous texture, a bright area, a light source attached to a surgical instrument, and/or another identifiable feature that may be recognized by suitable analysis performed by the computer 304. The feature of interest may also be a marking of a region of interest on a surgical instrument. The computer 304 may store information about characteristics of surgical instruments for use in recognizing the feature of interest.

The method of FIG. 8 includes determining a relative positioning between the feature of interest and an OCT scan location (step 802). For example, the processor 308 and memory 310 of the computer 304 may process captured images to recognize or identify the feature of interest. In addition, the processor 308 and memory 310 may be configured to determine a position and orientation of the feature of interest with respect to the OCT scan location. For example, an image capture including the feature of interest may be calibrated or coordinated with an OCT scan. It may be determined whether the position of the feature of interest is co-located with coordinates of a most recent OCT scan.

Methods for determining an OCT scan location include those based on determining the absolute position of a pre-set OCT scan location with respect to the field of view of the surgical microscope and attached imaging devices attached to it, as well as those based on on tracing the history of previous scan locations since an absolute determination was last made. Methods for determining the absolute OCT scan location include performing careful calibration between the OCT scanner unit and the surgical microscope field of view prior to surgery, utilizing an infrared camera mounted to the surgical microscope which is capable of directly viewing the OCT beam, and localizing the OCT scan location based on visualizing a common feature of interest in both the OCT image and the video image simultaneously.

The method of FIG. 8 includes controlling capture of an OCT image at a set position relative to the feature of interest based on the relative positioning (step 804). For example, the computer 304 may control the OCT unit 200 to acquire one or more OCT scans based on the relative positioning. The computer 304 may determine the difference between the position of the feature of interest and a current OCT scan location for adjusting the OCT scan location to acquire scans at the position of the feature of interest.

The OCT scans acquired at a position of a feature of interest may be B-scans having a predetermined scan pattern. For example, the predetermined scan pattern may be a single B-scan such as the scan pattern shown in FIGS. 5A and 5B. In the example of FIGS. 5A and 5B, the scan patterns have a particular position and orientation with respect to the surgical instrument 500. In examples of FIGS. 5A-5F, the computer 304 determines both a position and orientation of the instrument such that the scan patterns can have a particular position and orientation with respect to the surgical instrument. In other examples, the scan pattern may be a pair of B-scans (e.g., the scan pattern of FIG. 5C), or a multiple B-scans oriented in a radial or raster scanning pattern (e.g., the scan patterns of FIGS. 5D-5F).

In an embodiment, capture of an OCT image may be controlled based on a surgical procedure or an instrument being used during a surgical procedure. For example, the computer 304 shown in FIG. 3 may determine a type of surgical procedure or surgical instrument. An operator of the computer 304 may, for example, enter input into the user interface 314 (e.g., keyboard or mouse) for indicating a type of surgical procedure being performed. In another example, an operator may enter input into the user interface 314 for indicating a type of surgical instrument being used during a surgical procedure. Based on the identified surgical procedure and/or surgical instrument, the OCT unit 200 may be controlled to capture one or more OCT images having a particular pattern within a predetermined area. The OCT images may be captured within an area with respect to a position of the surgical instrument and based on the surgical procedure. For example, it may be desired to have a particular view of a surgical site and an instrument within the surgical site in the case of a particular surgical procedure and/or surgical instrument being used.

Referring again to FIG. 8, the steps 800, 802, and 804 may be repeated for tracking OCT image acquisition to the feature of interest. For example, subsequent to B-scans being captured of a feature of interest, a position of the feature of interest may be determined again such that OCT image acquisition can be controlled to track the feature of interest. In this way, for example, an OCT unit can be controlled to track a surgical instrument or a tissue feature for facilitating view of these features by a surgeon.

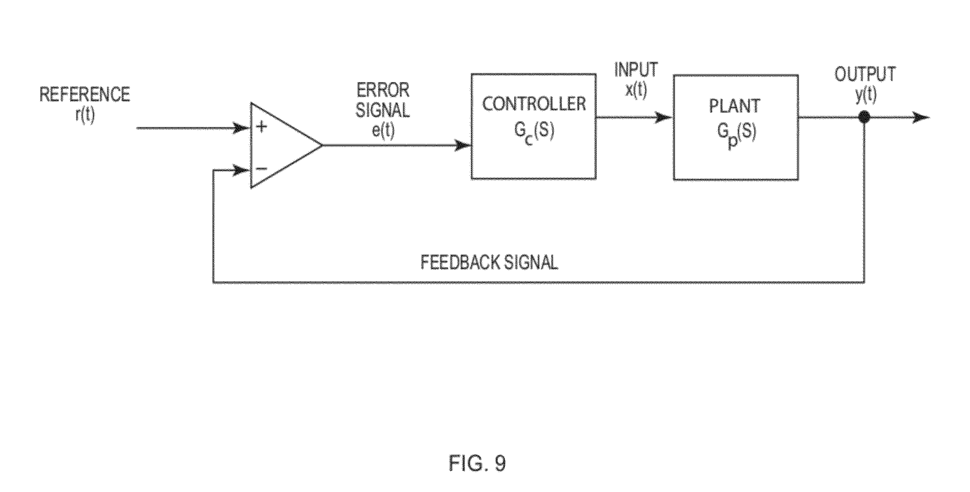

In accordance with an embodiment for tracking a feature of interest using OCT, FIG. 9 illustrates a schematic diagram of an example model for image feature tracking. The model represents a closed-loop control system that may be implemented by the computer 304 shown in FIG. 3 or any other suitable controller. Referring to FIG. 9, the input to the control system is a reference signal r(t) corresponding to a position of a feature of interest within the surgical field, such as a surgical instrument. The output y(t) corresponds to an OCT scan location. The error signal e(t) represents the difference between the position of the feature of interest and an OCT scan location, which in a closed-loop tracking system is desired to be minimized. The difference error signal e(t) is fed into the controller Gc(s), which applies well known control algorithms to construct the signal x(t) which is optimized to drive the OCT scanner unit represented as the plant Gp(s) to obtain OCT images at a position which minimizes the difference between the OCT scan position and the position of the feature of interest. The variable t represents time, and the variable s represents complex frequency as is common in linear systems and control theory. The purpose of the control system is to drive the OCT scanner unit, such as the OCT unit 200 shown in FIG. 3, to track OCT image acquisition to the feature of interest.

The reference signal r(t) may represent a specified feature of a surgical instrument, which may be detected or otherwise identified by the computer 304 or any suitable instrument localizing sub-system. The computer 304 may identify a position and orientation of the instrument with sufficient resolution to direct the system to perform OCT imaging in its vicinity. In an embodiment, the computer 304 may extract the instrument position information by computerized analysis of the color video camera signal which may be configured to provide a duplicate of the surgeon's view through the surgical microscope. In an example, the video camera image may be aligned and calibrated with respect to the OCT scanner coordinate system, and control provided for such alignment to be maintained throughout surgery. Live image processing can be performed on the video image signal to recognize a feature of interest, such as a surgeon's instrument. The instrument may be configured with features that are distinct from the rest of the surgical view, such as straight edges, non-tissue colors, homogeneous texture in its body, or brightly reflective points. The computer 304 may implement a computer vision algorithm that recognizes these features and estimates the position and orientation of a tool in view. For example, such an algorithm may limit the search region by observing a limited square view in the most recently acquired image frame of the camera. In this example, a limited square is centered on the initially-estimated tool location, beginning either in the center of the field or by a user who is simultaneously operating the tracking software computer. Within the limited square, the location of the peak intensity is determined, and the limiting square is re-centered at this location for the next image frame. Because the surgical instruments may have brightly reflecting tips, the brightest part of the view will be at the tool tip in this case.

In another embodiment, an infrared (IR) sensitive video camera may be used for capturing images. As a result, visualization of the OCT illumination may be provided, rather than the visible illumination. This configuration may allow the system to verify that the beam is actually coincident with the location of interest, such as a surgical instrument tip. In this case, a computer vision algorithm may be configured to recognize the known and expected shape of the beam focused on the sample. An alternative embodiment uses an additional imaging modality which provides a higher quality en-face view of the surgical field than a color video camera, such as a scanning laser ophthalmoscope (SLO) which can be built into the OCT system for simultaneous OCT/SLO imaging. An alternative embodiment may use an aspect of the OCT imaging system itself to localize the instrument position. For example, surgical tools constructed of metal or plastic may be identified in OCT B-scans by their specific reflectance or shadowing profile. Since OCT B-scans are highly localized in the direction perpendicular to their scan direction, they would not provide any localization information in that dimension. However, the instrument may be equipped with a predetermined marking (such as a divot) which may be recognizable in an OCT B-scan such that a computer vision algorithm can distinguish between the subject tissue and features of a surgical instrument in the cross sectional view of a B-scan. To find the instrument in the view within a view such as a surgical site view, the OCT system may scan in a “search mode” until the instrument is recognized. The OCT system may determine whether a feature of interest is not contained within captured image, and capture images of a different area of a view to search for the instrument within the view in response to determining that the image is not contained within the captured image. Once the instrument is recognized, the system may enter a “locked-on” mode, such as the example method shown in FIG. 8 which always keeps scanning near the instrument. To remain locked on, the OCT system may regularly scan in locations just outside the plane of a central B-scan to check if the instrument has been displaced. Fast scanning and tight search range may be used for continuing to lock onto the instrument. Once the instrument has escaped the view, the OCT system may return to the search mode. These processes may be implemented by, for example, the computer 304 controlling the OCT unit 200.

Various surgical instruments may be tracked in accordance with embodiments of the present disclosure. These instruments may be used in OCT-imaged surgical procedures as disclosed herein. Example surgical instruments that may be tracked include, but are not limited to, scalpels, forceps, scrapers, scissors, picks, vitrectomy tips, and other instruments and tools in common use in microsurgery including vitreoretinal surgery. These instruments may include a body having a predefined shape for improving capture of OCT images of nearby tissue during a surgical procedure. Examples body shapes include, but are not limited to, flat or sharp-edged shapes. Such shapes may have varying reflectance and shadowing patterns. The body of the instrument may be constructed from a variety of materials, including metals, plastics, polyamide, silicone, and composites. Other example materials include clear or tinted plastic, polymers, glass, ceramics, or other materials to allow control of the transmission and reflectance of an OCT beam. Such instruments may be compatible with intrasurgical OCT imaging as described herein and optimized for such factors as transparency to OCT light, or having localized or distributed reflecting material embedded within them for enhanced visibility with OCT. Additionally, instruments may be specifically designed or modified for compatibility with the tool localizing sub-system as disclosed herein. For example, instruments may be designed for detection by a color video camera, IR camera, or SLO image and may be specially marked in a certain pattern of one or more markings or color which uniquely identifies the instrument position and orientation in the en-face camera view. In an example, the body of an instrument may have multiple markings on its surface that have a predefined spacing for use in determining a distance in a capture OCT image including the markings. Such instruments may also be modified or designed to have a small light source or emitter, such as a light emitting diode or optical fiber tip embedded within them, so that the emitted light can be detected and localized by the en-face camera image analysis software. In an example, an instrument body may define an interior channel such that a optical fiber may be embedded within it and connected to a light source at one end. The opposing end of the fiber optic may be positioned to terminate at a pre-defined location within the surgical instrument for view from an exterior of the body such that when the light source is activated, light is emitted and viewable for tracking the instrument. Further, all or a portion of the body or the surface may be modified to selectively increase or decrease OCT transmission and reflectance, such as through abrading or diamond dusting the surface or alternatively embedding reflectors within the body to increase reflectance on OCT and increase visualization of the instrument. Modification on the surfaces can be performed to decrease reflectivity and further improve visibility of underlying structures. In an example, an instrument tip may have portions of reflective and non-reflective material to optimize the view of surrounding structures while at the same time, maintaining optimal view of the instrument for OCT control or for view by a surgeon.

The error signal e(t) may be a difference between the reference signal r(t) and output signal y(t). A controller sub-system G_{c}(s) may employ predetermined information about the characteristics of the instrument localizing sub-system and plant G_{P}(s), along with suitable feedback control algorithms, to process the error signal e(t) to produce an input signal x(t) which directs the OCT scanner unit 200, represented by the plant Gₚ(s), to perform OCT imaging at the location y(t) which the controller Gc(s) causes to track the feature of interest. The OCT scan unit 200 comprises the plant which is controlled by the computer 304 implementing the model of FIG. 9 to capture a desired OCT scan pattern, such as any of the scan patterns illustrated in FIGS. 5A-5F.

The OCT scan unit 200 may be represented by the plant Gₚ(s) shown in FIG. 9. In an example, control unit 200 may be any suitable OCT scanning apparatus. OCT scanning apparatus may include orthogonal galvanometer-driven optical mirrors. The OCT scanning apparatus may implement its own spatial coordinate system, governed by its attachment to the surgical microscope, which may be aligned and calibrated to the tool localizing sub-system. The OCT scanning apparatus may have a known transfer function which converts the plant input signal x(t) to the output signal y(t) corresponding to the desired imaging location, taking into account such factors as any gain, offset, or temporal delay inherent to the scanning hardware and electronics which control it.

In the example of FIG. 3, the OCT scanning unit 200 and the camera 302 are synchronized to provide complementary information. The camera 302 may be a wide field of view, video-rate surgical video camera, a scanning laser ophthalmoscope, or any other high frame rate ophthalmic camera. Analysis of images captured by the camera 302 may provide rough estimates of the surgical tool position (azimuthal and lateral) on the patient eye. Other sensor input is the limited field of view, low-frame rate SDOCT B-scan data captured by the OCT scanning unit 200. The B-scan data can provide topographic information about a surgical site, such as sub-retinal structures. To simplify the computational complexity of image registration in this example, the OCT scanning unit 200 and the camera 302 are physically connected, resulting in a synchronized monotonous motion in the corresponding images. The connectivity condition may be removed by using faster computer processors. Any suitable multi-sensor image alignment algorithm, including mutual information-based registration techniques or vessel alignment based registration method, such as the method disclosed in “Enhanced Video Indirect Ophthalmoscopy (VIO) via Robust Mosaicing,” by R. Estrada, C. Tomasi, M. T. Cabrera, D. K. Wallace, S. F. Freedman, S. Farsiu. (BioMedical Optics Express, Vol. 2, Iss. 10, pp. 2871-2887, October 2011), the disclosure of which is incorporated herein by reference, may be implemented to parametrically register the SDOCT generated SVP image to the en face video sequence. To improve registration estimation accuracy and speed, calibration of the OCT unit 200 and the camera 302 may be performed accurately before the commencement of surgery. Then, during the surgery a computationally-efficient projective operation relates the position of the surgical tool on the en face video camera, to approximate position on the SDOCT camera view.

Various mechanisms can be utilized for guiding an SDOCT scanner to scan a feature of interest. In an example, a smooth sub-millimeter (local) displacements of a surgical instrument on the SDOCT B-scans may be tracked. The trajectory of the surgical instrument may be predicted and its motion trajectory locked onto in a Kalman-filtering framework. An en face video camera with large field of view may be used to track and compensate for large displacements, such as when a surgeon moves the instrument a large distance very quickly.

To fuse information from multiple scans, several repeated fast sparse 3-D scans may be rapidly captured. Subsequently, captured scan that have been affected by motion artifacts may be removed. Next, the artifact-free scans may be fused, a dynamically updated high-resolution 3-D scan generated.

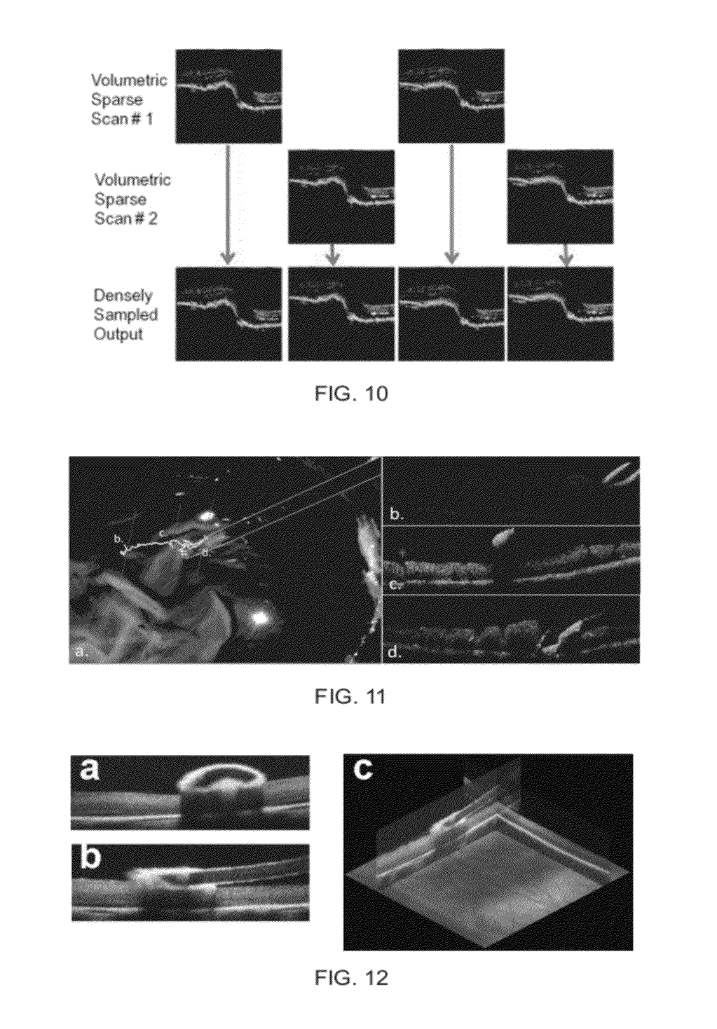

At each time point, of the very large number of sparse scans that the MIOCT system captures, sparsely sampled volumetric scans may be captured with a significantly lower number of B-scans than the target resolution. The image fusion time-window, i.e. the optimal number of volumes to be fused (N), may be selected based on a maximum preset threshold which can be decreased in case of scene change. Since the number of frames in each sequence (K) may be relatively small, each scan is captured very fast. For this reason, it may be assumed that some of these sequences will be less affected by the abrupt patient motion. Such sequences may be detected, reordered, and interlaced for creating a densely sampled, artifact-free representation of a surgical view, such as the retina. FIG. 10 illustrates images in which two sparsely sampled sequences are fused together creating a dense representation of the underlying pathological structures. Referring to FIG. 10, fusing (or interlacing) multiple sparsely sampled scan sequences can create an azimuthally higher resolution volume of B-scans. This process may be repeated for each time point in the output video. A 3-D extension of a 2-D Kalman filtering may be used to create a smoothly changing (unless there is a rapid change in the eye location) dynamic high-resolution volume of a feature of interest, such as an eye.

In an example, a computer, such as the computer 304 shown in FIG. 3, may implement a digital filter. In an example, a most recent tip location, represented as X(z), may act as a computed result of an image processing algorithm. This input may be tested against the last result of the actual scanning position Y(z)*z⁻¹, and the error signal may be scaled to define a new position. Depending on the gain, k, which determines the speed of response, the scanner may move toward the desired position over multiple frames in a smooth fashion. This gradual motion may improve stability of the tracking and reduce high-frequency fluttering of the galvanometer scanners. Computed new tracking offset voltages may be generated by a linked digital to analog converter card, and summed with an SDOCT system. This alleviated signal/image processing burden from the SDOCT computer, which operated separately from any tracking input.

FIG. 11 depicts various images of example data obtained from simulated surgery on an excised porcine eye with the anterior segment removed. Particularly, FIG. 11 shows fundus and OCT images during a simulated surgical maneuver. Image (a) shows a grayscale image of the fundus view is shown with the outline of the surgical scraper instrument outlined. A small white box with a black cross is superimposed on the tool tip in the live image. This snapshot was taken after the scraper was dragged from left to right approximately 6 mm along the surface of the retina, therefore, the box highlights the final position of the tip. Another line in image (a), indicates the calculated tool tracking position throughout the motion. Continuous B-scan images (1024×1000 pixels) were recorded by an SDOCT system with 17 B-scans per second and 50 μs integration time per A-scan. The illuminating beam power was limited to 700 μW. Images (b)-(d) show three B-scans acquired at the lateral positions indicated by points b, c, and d in image (a). As visible in image (b), the porcine retina had several detachments and folds, however the surgical tool tip was clearly visible as a bright object intersecting the retina from above right. In image (c), the tool tip is visible in the center, over the retina and causing partial shadowing of the retina. The tip appears bright due to diamond dust particles embedded in the silicone tip which strongly scatter OCT light. Also, a small, but distinct frame-breaking artifact in the OCT scan in image (c) is highlighted by a star. This discontinuity is due to a short but rapid shift in the scanner position mid-scan. In image (d), the B-scan is sectioning the transparent silicon portion of the instrument tip, which appears semicircular, and is pushing down, and causing retinal deformation. The silicone is mostly transparent to the OCT beam, and therefore retinal features below the tip are visible. The three B-scans (image s(b)-(d)) shown are separated in time by approximately 5 seconds each. The algorithm tracked the tip consistently despite the two nearby glare artifacts due to the room lighting, both visible in image (a).

FIG. 12 illustrates two orthogonal B-scans, images (a) and (b), acquired using the scan pattern shown in FIG. 5C during simulated porcine surgery. In image (c) of FIG. 12, the orthogonal B-scans are rendered in their correct orientation in 3D above an image of the retinal surface.

In accordance with embodiments of the present disclosure, a surgical microscope system may be configured with a heads-up display (HUD) for providing surgical information to a surgeon. A HUD may integrate visualization of OCT images and/or other surgical information into the optical viewports of a surgical microscope, such as the MIOCT system 100 shown in FIG. 1. In this way, simultaneous display of real-time cross-section OCT imaging may be provided with direct viewing of surgical sites and instruments. Further, various surgical information may be presented to an operator via the user interface 314 shown in FIG. 3, such as audibly via a speaker.

Information displayed by the HUD may be viewed through an ocular eyepiece unit, such as the oculars 308 shown in FIG. 3. In an embodiment, a HUD may be implemented by attaching two assistant viewing extensions to the surgical microscope. Each assistant viewing extension may include a video camera port in addition to the assistant ocular eyepieces. On one assistant arm, the video camera can be used for archival purposes and also for providing the video fundus image view required for the SDOCT scan-directing algorithms described below. However, in the second assistant arm, beamsplitter leading to the camera port may be reversed, and the camera may be replaced with a similarly sized high-resolution organic light-emitting diode (OLED) or other type array connected as an auxiliary monitor for the SDOCT computer. This allows an observer on the second assistant arm to view whatever is displayed on the OLED display overlaid with optical view the surgical microscope is transmitting. The SDOCT display may be displayed in a “dark” area outside the primary microscope view (i.e., the dark area outside the center of view of a conventional microscope eyepiece, normally rendered black by the presence of an aperture stop).

Referring to the example of FIG. 3, the computer 304 may operate as a user interface controller for controlling a HUD 316. For example, the computer 304 may determine surgical information associated with a surgical site image projected for view through an ocular eyepiece unit. Further, the computer 304 may control the HUD 316 to display the surgical information. In an example, surgical information may include an indication of a distance between features (e.g., a surgical instrument and tissue feature such as a retinal layer) within a surgical site image. For example, the computer 304 may be configured to identify a surgical instrument and tissue feature in an image projected to a surgeon thought the ocular eyepiece unit. Subsequent to the identification, the computer 304 may analyze the positioning to determine a distance between the instrument and tissue feature. The computer 304 may control the HUD 316 to display a number or any other indicia of the distance. In another example, the surgeon may select among different data for display via the HUD 316. Another example of feedback includes vibratory feedback measurement guide displays.

It is noted that many of the examples provided herein relate to ophthalmic surgery; however, the systems, methods, and instruments disclosed herein may also be applied to other types of surgeries or any other suitable procedure. Example surgeries include, but are not limited to, neurosurgery, breast surgery, dermatologic procedures, otolaryngologic procedures, such as tympanic membrane, or any other surgeries requiring precise maneuvers to small subsurface structures (e.g., Schlemm's canal) visible on OCT or other imaging equipment.

The various techniques described herein may be implemented with hardware or software or, where appropriate, with a combination of both. Thus, the methods and apparatus of the disclosed embodiments, or certain aspects or portions thereof, may take the form of program code (i.e., instructions) embodied in tangible media, such as floppy diskettes, CD-ROMs, hard drives, or any other machine-readable storage medium, wherein, when the program code is loaded into and executed by a machine, such as a computer, the machine becomes an apparatus for practicing the presently disclosed subject matter. In the case of program code execution on programmable computers, the computer will generally include a processor, a storage medium readable by the processor (including volatile and non-volatile memory and/or storage elements), at least one input device and at least one output device. One or more programs may be implemented in a high level procedural or object oriented programming language to communicate with a computer system. However, the program(s) can be implemented in assembly or machine language, if desired. In any case, the language may be a compiled or interpreted language, and combined with hardware implementations.

The described methods and apparatus may also be embodied in the form of program code that is transmitted over some transmission medium, such as over electrical wiring or cabling, through fiber optics, or via any other form of transmission, wherein, when the program code is received and loaded into and executed by a machine, such as an EPROM, a gate array, a programmable logic device (PLD), a client computer, a video recorder or the like, the machine becomes an apparatus for practicing the presently disclosed subject matter. When implemented on a general-purpose processor, the program code combines with the processor to provide a unique apparatus that operates to perform the processing of the presently disclosed subject matter.

Features from one embodiment or aspect may be combined with features from any other embodiment or aspect in any appropriate combination. For example, any individual or collective features of method aspects or embodiments may be applied to apparatus, system, product, or component aspects of embodiments and vice versa.

While the embodiments have been described in connection with the various embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed:

1. A method for optical coherence tomography (OCT) imaging, the method comprising:
receiving a plurality of OCT B-scans of a field of view area that includes an instrument; and
applying spatial compounding to the B-scans to generate an OCT image of the area of the field of view area.

2. The method of claim 1, wherein the instrument is a surgical instrument.

3. The method of claim 1, wherein the B-scans have a scan pattern aligned substantially within an axis of the instrument.

4. The method of claim 1, wherein the B-scans have a pattern with a long-axis aligned substantially with an axis of the instrument and a short-axis substantially collinear with the instrument.

5. The method of claim 1, wherein applying spatial compounding the B-scans comprises combining the B-scans to generate the OCT image.

6. The method of claim 5, wherein combining the B-scans comprises averaging the B-scans.

7. The method of claim 5, wherein combining the B-scans comprises performing a weighted average of the B-scans.

8. The method of claim 1, further comprising controlling an OCT unit to capture the OCT B-scans.

9. The method of claim 1, further comprising controlling an OCT unit to capture OCT B-scans at a lateral spacing of a predetermined distance.

10. The method of claim 1, further comprising controlling the OCT unit to capture OCT B-scans at temporal spacing of a predetermined amount of time.

11. An optical coherence tomography (OCT) system comprising:
an OCT unit configured to receive a plurality of OCT B-scans of a field of view area that includes an instrument; and
a computing device configured to apply spatial compounding to the B-scans to generate an OCT image of the area of the field of view area.

12. The system of claim 11, wherein the instrument is a surgical instrument.

13. The system of claim 11, wherein the B-scans have a scan pattern aligned substantially within an axis of the instrument.

14. The system of claim 11, wherein the B-scans have a pattern with a long-axis aligned substantially with an axis of the instrument and a short-axis substantially collinear with the instrument.

15. The system of claim 11, wherein the computing device is configured to combine the B-scans to generate the OCT image.

16. The system of claim 15, wherein the computing device is configured to average the B-scans.

17. The system of claim 15, wherein the computing device is configured to perform a weighted average of the B-scans.

18. The system of claim 11, wherein the computing device is configured to control the OCT unit to capture the OCT B-scans.

19. The system of claim 11, wherein the computing device is configured to control the OCT unit to capture OCT B-scans at a lateral spacing of a predetermined distance.

20. The system of claim 1, wherein the computing device is configured to control the OCT unit to capture OCT B-scans at temporal spacing of a predetermined amount of time.

21. A method for optical coherence tomography (OCT) image capture, the method comprising:
determining a location of a feature of interest within an operative field;
determining a relative positioning between the feature of interest and an OCT scan location; and
controlling capture of an OCT image at a set position relative to the feature of interest based on the relative positioning.

22. The method of claim 21, wherein the feature of interest is one of a surgical instrument or a tissue feature within an operative field, and
wherein the method further comprises recognizing the feature of interest within the operative field.

23. The method of claim 21, wherein the feature of interest is one of a straight edge, a color, a marking, a homogenous texture, a recognizable shape of an area of a surgical instrument, a bright area of a surgical instrument, or a light source attached to a surgical instrument within an operative field, and
wherein the method further comprises recognizing the feature of interest within the operative field.

24. The method of claim 21, further comprising receiving at least one image including the feature of interest, the at least one image comprising one of video images, an OCT B-scan, an OCT summed voxel projection (SVP) image, or a scanning laser ophthalmoscopy (SLO) image.

25. The method of claim 21, wherein determining the location of the feature of interest comprises determining the location of the feature of interest by use of one of an ultrasound technique, a computed tomography technique, a magnetic resonance imaging technique, and a radiofrequency triangulation technique.

26. The method of claim 21, wherein determining a relative positioning comprises determining a position and orientation of the feature of interest with respect to the OCT scan location.

27. The method of claim 21, wherein the feature of interest is a marking of a region of interest on a surgical instrument.

28. The method of claim 21, further comprising receiving at least one image that corresponds to a display image.

29. The method of claim 21, wherein controlling capture of the OCT image comprises controlling an OCT unit to capture B-scans having a predetermined scan pattern.

30. The method of claim 29, wherein the predetermined scan pattern is a single B-scan, a pair of b-scans oriented orthogonal to each other, or a plurality of B-scans oriented in a radial or raster scanning pattern.

31. The method of claim 29, wherein the feature of interest is a surgical instrument, and
wherein controlling capture of the OCT image comprises controlling an OCT unit to capture a B-scan that is aligned with an axis of the surgical instrument.

32. The method of claim 21, wherein the feature of interest is a surgical instrument,
wherein the method further comprises determining a type of surgical procedure, and
wherein controlling capture of the OCT image comprises controlling capture of the OCT image within a predetermined area with respect to a position of the surgical instrument and based on the type of the surgical procedure.

33. The method of claim 21, wherein the feature of interest is a surgical instrument,
wherein the method further comprises determining a type of the surgical instrument, and
wherein controlling capture of the OCT image comprises controlling capture of the OCT image within a predetermined area with respect to a position of the surgical instrument and based on the type of the surgical instrument.

34. The method of claim 21, further comprising determining an orientation of the feature of interest, and
wherein controlling capture of the OCT image comprises controlling capture of the OCT image based on the orientation of the feature of interest.

35. The method of claim 21, further comprising controlling capture of a plurality of OCT images for searching for the feature of interest within an operative field.

36. The method of claim 21, further comprising:
determining whether the feature of interest is not contained within at least one of multiple images captured of an operative field; and
in response to determining that the feature of interest is not contained within the at least one of the multiple images, controlling capture of a plurality of second images captured of a different area of the operative field to search for the feature of interest within the operative field.

37. The method of claim 21, further comprising repeating the determining steps and the controlling step for tracking OCT image acquisition to the feature of interest.

38. A system for optical coherence tomography (OCT) image capture, the system comprising:
at least one processor configured to:
determine a location of a feature of interest within an operative field;
determine a relative positioning between the feature of interest and an OCT scan location; and
control an OCT unit to capture of an OCT image at a set position relative to the feature of interest based on the relative positioning.

39. The system of claim 38, wherein the feature of interest is one of a surgical instrument or a tissue feature within an operative field, and
wherein the at least one processor is configured to recognize the feature of interest within the operative field.

40. The system of claim 38, wherein the feature of interest is one of a straight edge, a color, a marking, a homogenous texture, a recognizable shape of an area of a surgical instrument, a bright area of a surgical instrument, or a light source attached to a surgical instrument within an operative field, and
wherein the at least one processor is configured to recognize the feature of interest within the operative field.

41. The system of claim 38, wherein the at least one processor is configured to receive at least one image including the feature of interest, the at least one image comprising one of video images, an OCT B-scan, an OCT summed voxel projection (SVP) image, or a scanning laser ophthalmoscopy (SLO) image.

42. The system of claim 38, wherein the at least one processor is configured to determine the location of the feature of interest by use of one of an ultrasound technique, a computed tomography technique, a magnetic resonance imaging technique, and a radiofrequency triangulation technique.

43. The system of claim 38, wherein the at least one processor is configured to determine a position and orientation of the feature of interest with respect to the OCT scan location.

44. The system of claim 38, wherein the feature of interest is a marking of a region of interest on a surgical instrument.

45. The system of claim 38, wherein the at least one processor is configured to receive at least one image that corresponds to a display image.

46. The system of claim 38, wherein the at least one processor is configured to control the OCT unit to capture B-scans having a predetermined scan pattern.

47. The system of claim 46, wherein the predetermined scan pattern is a single B-scan, a pair of b-scans oriented orthogonal to each other, or a plurality of B-scans oriented in a radial or raster scanning pattern.

48. The system of claim 46, wherein the feature of interest is a surgical instrument, and
wherein the at least one processor is configured to control the OCT unit to capture a B-scan that is aligned with an axis of the surgical instrument.

49. The system of claim 38, wherein the feature of interest is a surgical instrument, and
wherein the at least one processor is configured to:
determine a type of surgical procedure; and
control the OCT unit to capture of the OCT image within a predetermined area with respect to a position of the surgical instrument and based on the type of the surgical procedure.

50. The system of claim 38, wherein the feature of interest is a surgical instrument, and
wherein the at least one processor is configured to:
determine a type of the surgical instrument; and
controlling the OCT unit to capture of the OCT image within a predetermined area with respect to a position of the surgical instrument and based on the type of the surgical instrument.

51. The system of claim 38, wherein the at least one processor is configured to:
determine an orientation of the feature of interest; and
control capture of the OCT image based on the orientation of the feature of interest.

52. The system of claim 38, wherein the at least one processor is configured to control the OCT unit to capture of a plurality of OCT images for searching for the feature of interest within an operative field.

53. The system of claim 38, wherein the at least one processor is configured to:
determine whether the feature of interest is not contained within at least one of multiple images captured of an operative field; and
in response to determining that the feature of interest is not contained within the at least one of the multiple images, control the OCT unit to capture of a plurality of second images captured of a different area of the operative field to search for the feature of interest within the operative field.

54. The system of claim 38, wherein the at least one processor is configured to repeat the determining functions and the controlling function for tracking OCT image acquisition to the feature of interest.

55. A surgical microscope system comprising:
a heads-up display (HUD);
an ocular eyepiece unit having the HUD integrated therein for display via the ocular eyepiece unit; and
a user interface controller configured to:
determine surgical information associated with a surgical site image projected for view through the ocular eyepiece unit; and
control the HUD to display the surgical information.

56. The surgical microscope system of claim 55, wherein the surgical information includes an indication of a distance between features within the surgical site image, and
wherein the controller is configured to determine the distance between features within the surgical site image.

57. The surgical microscope of claim 56, wherein the features include a surgical instrument and a tissue feature.

58. The surgical microscope of claim 57, wherein the controller is configured to identify the surgical instrument and the tissue feature.

59. The surgical microscope of claim 55, wherein the controller is configured to control a user interface device based on the surgical information.

60. The surgical microscope of claim 59, wherein the controller is configured to:
determine a distance between features within the projected image; and
control the user interface device to present an indication of the distance.

61. The surgical microscope of claim 55, wherein the controller is configured to:
receive user input to select the surgical information for display; and
control the HUD to display the selected surgical information.

62. A surgical instrument for use in optical coherence tomography (OCT)-imaged surgical procedures, wherein the surgical instrument comprises a body having a predefined shape for improving capture of OCT images of nearby tissue during a surgical procedure.

63. The surgical instrument of claim 62, wherein the predefined shape comprises at least one marking configured for identification in the captured OCT images.

64. The surgical instrument of claim 63, wherein the at least one marking comprises multiple markings having a predefined spacing for use in determining a distance in a captured OCT image including the markings.

65. The surgical instrument of claim 62, wherein a portion of a surface of the body has a predefined color for identification of the surface in the captured OCT images.

66. The surgical instrument of claim 62, further comprising a light emitter attached to the body.

67. The surgical instrument of claim 66, wherein the light emitter comprises a light emitting diode (LED).

68. The surgical instrument of claim 66, wherein the body defines an interior, and
wherein the light emitter comprises a fiber optic configured for connection to a light source at one end and having an opposing end positioned for view from an exterior of the body.
